(19) United States
(12) Patent Application Publication
(10) Pub. No.: US 2025/0009965 A1
(43) Pub. Date: Jan. 9, 2025

(54) APPARATUS AND METHOD FOR SCALABLE FLUID DELIVERY

(71) Applicant: INDIAN INSTITUTE OF SCIENCE, Bangalore (IN)

(72) Inventors: Deval KARIA, Bangalore (IN); Manish ARORA, Bangalore (IN)

(73) Assignee: INDIAN INSTITUTE OF SCIENCE, Bangalore (IN)

(21) Appl. No.: 18/712,469

(22) PCT Filed: Nov. 22, 2022
(86) PCT No.: PCT/IB2022/061264

§ 371 Date: May 22, 2024

(30) Foreign Application Priority Data
Nov. 23, 2021 (IN) 202141053960

(51) Int. Cl. A61M 5/145

(52) U.S. Cl. CPC A61M 5/1452; A61M 2005/14533

(57) ABSTRACT

Present disclosure generally relates to devices and methods for conveying small amounts of fluids, and particularly relates to apparatus and method for scalable fluid delivery to achieve minimal motion of syringe plunger using multiple kinematic chain elements/pathways actuated by a single Motion Actuating Device (MAD). The scalable fluid delivery apparatus provides first kinematic pathway corresponding to high linear resolution mode of Piston Drive Member (PDM) and second kinematic pathway corresponding to low linear resolution mode of PDM. The first kinematic pathway includes final step of delivering fluid comprised in syringe, upon pushing plunger, and second kinematic pathway includes final step of performing forward motion or reverse motion of PDM. Forward motion, or reverse motion of PDM is for priming barrel and removable fluid delivery tube or to revert the PDM to an initial position, respectively, or to deliver the fluid.

### FIELD OF INVENTION

The embodiments of the present disclosure generally relate to devices and methods for conveying small amounts of fluids. More particularly, the present disclosure relates to apparatus and method for scalable fluid delivery via a minimal motion of a syringe plunger using multiple kinematic chain elements and/or pathways actuated by a single Motion Actuating Device (MAD).

### BACKGROUND OF THE INVENTION

The following description of related art is intended to provide background information pertaining to the field of the disclosure. This section may include certain aspects of the art that may be related to various features of the present disclosure. However, it should be appreciated that this section be used only to enhance the understanding of the reader with respect to the present disclosure, and not as admissions of prior art.

In general, a scalable mechanism for fluid finds primary application in infusion devices. A global market for such devices may be USD19.60 billion in value terms in 2020 and is expected to grow at a CAGR of 7.21% and reach USD29.40 billion by 2026. The Indian market may be significant and may be expected to reach USD572 million by 2024. This may be primarily driven by the growing geriatric population suffering from various chronic diseases. Despite such promising forecasts, the high cost of such devices may be expected to hamper growth in all markets. Based on the end user, this market can be fragmented into hospitals/clinics, ambulatory care settings, and others. Each end user may have different requirements of delivery resolution and size constraints. The scalable and affordable nature of the mechanism allows it to be adapted for multiple such users (in terms of resolution, size) and markets (in terms of price points, regulations). The potential for an affordable solution may be significant, particularly in India with increased awareness, rising number of patients and lack of options being primary contributors. Generally, drug delivery via micro-motion may be primarily achieved using a high precision micro-motor set (motor+gearbox+encoder) to achieve the requisite linear motion, a Shape Memory Alloys (SMAs) to actuate a ratchet-lead screw based kinematic chain, and a peristaltic device(s) which may rely on a flexible tubing and a rotary actuator for compression. The high precision micro-motor set may be reliable, however, may also be prohibitively expensive and rely on a planetary gear train to achieve speed reduction. The motor diameter may generally be in the range of 8-12 millimetre (mm), making the internal gear train even smaller. Such size reduction in the internal gear train may lead to higher cost. Further, the SMAs while being cost effective, may have a limited cycle life. Typically, with repeated actuation the SMAs may not last for more than 10,000-15,000 cycles, and the response characteristics of the SMAs may also change with time. Accordingly, the SMAs may only be suitable for disposable devices. Thereafter, the peristaltic micro-pumps such as the micro-motor-based systems may be prohibitive in terms of cost. In addition, it may be extremely difficult to achieve the required precision of volume delivery and durability for a device which may be used for 3-4 years.

Conventional apparatus discloses a variable volume, shape memory actuated dispensing pump as shown in FIG. 1A. A pump system 100A is shown in the inactive state in FIG. 1A. The pump body comprises a case 101, a top cap 102, and a plunger cap 103. Within the pump is a plunger 104 that is normally (in the inactive state) held against the plunger cap 103 by a plunger bias spring 105. Similarly, an overload piston 106 is normally (in inactive state) held against the top cap 102 by an overload piston spring 107 which is stronger (has a higher spring constant k) than the plunger bias spring 105. The plunger 104 is connected to the overload piston 106 by a shape memory alloy wire 108 which contracts when heated by a pulse or pulses of current flowing from the V+ 109 contact to the V− 110 contact through the shape memory alloy wire 108 where the V− 110 contact may be the system ground (GND) reference. The pumping system 100A may include a chamber, a piston 106 within the chamber, a shape memory element within the chamber that changes length when a current flows through the element, the element moving the piston between a first and second physical limit, such that when the piston is at the first limit the chamber is occupied with liquid and when the chamber is at the second limit the chamber is substantially discharged of liquid; and a first switch at the first limit that indicates if the piston is at the first limit.

Another conventional apparatus 100B discloses a Packaged peristaltic micropump for controlled drug delivery application, as shown in FIG. 1B. This conventional apparatus discloses a device that may be a peristaltic pump designed for drug delivery. The conventional apparatus 100B as shown in FIG. 1B includes a Direct Current (DC) motor with encoder 112, a motor fixture 113, a casing/tube holder 114, a cam/roller 115, a bearing 116, and a space to fix a silicon tube 117. The operating principle of the conventional apparatus may be a single cam-based 115 pumping, consequently reducing the tubing size. It dispenses a drug at a constant flow rate and varying pressure, with considerations for precise flow control and fail-safe operation. Pump 100 may be capable of dispensing 82 μL for one complete rotation. Yet another conventional apparatus discloses a compact medical pump device 100C, which discloses a fluid infusion pump device 132 for infusing at least one fluid-medicament to the user, as shown in FIG. 1C. A medicament outlet conduit is coupled to a medicament reservoir 137 for infusing the medicament 134 into the user. An infusion needle is fluidly coupled to the medicament outlet conduit and the infusion needle is stored within a central axle. The medicament reservoir 137 is integrated with a rotary pump for providing a user thereof with a controlled quantity of fluid-medicament. The rotary pump includes at least one stationary arm and at least one rotating arm such that on operating the rotary pump fluid-medicament is impelled through an outlet channel of the reservoir 137 thus infusing the medicament into a user.

Further, a conventional lead screw delivery device 100D using reusable shape memory actuator drive as shown in FIG. 1D, includes a single shape memory alloy wire actuator to advance a lead screw 134, via a ratcheting mechanism 133. In one embodiment, a shape memory alloy wire 132 is operably connected to one of a pair of ratchet wheels 148, 150 and configured to drive incrementally the rotation of the connected ratchet wheel 148 via a contraction, which in turn drives the rotation of the other ratchet wheel 150 about a rotational axis which moves a lead screw 134 and advances a plunger 159 to dispense a liquid drug from a drug container. A drug delivery device using the shape memory alloy wire actuator in combination with the ratcheting mechanism 133 to incrementally rotate a shaft, a lead screw 134 or a sleeve provides for a more compact design. However, the conventional lead screw delivery device 100D may not be reversible and may likely to be designed for single-use, since the linear movement of the lead screw 134 cannot be reversed because a motor may not be used in the conventional lead screw delivery device 100D. Further, the delivery volume resolution of the conventional lead screw delivery device 100D may be dependent on the SMA wire 132.

Further, conventional infusion pump system 100E of a universal applicable structure for infusing a liquid into a patient or person is shown in FIG. 1E. The interior of the check valves and the pump housing component 172-3. A first check valve comprises a central circular cylindrical housing component 172-8 from which a frusto-conical top part 172-9 extends upwardly communicating with the inlet tube 172-1 and arresting an inlet filter element 172-10 at the transition between the frusto-conical top part 172-9 and the cylindrical housing component 172-3. The cylindrical housing component 172-3 comprises a central annular oral component 172-11 which is sealed off in the initial or non-active position by a downwardly deflectable sealing membrane 172-12. Provided the pressure below the sealing membrane 172-12 is lower than the pressure above the membrane 172-12, the membrane 172-12 is forced downwardly, allowing liquid to pass through the central aperture of the central annular component 172-11 and further through apertures 172-13 provided offset relatively to the centre of the sealing membrane 172-12. An infusion pump unit includes a housing sized to allow the pump unit to be carried as a portable unit. The housing has an inlet for establishing fluid communication from an external infusion bag 172-30 to the inlet and an outlet for establishing fluid communication to an infusion site. The housing contains a controllable pumping system for pumping fluid from the inlet to the outlet and a shape memory alloy actuator 172-7 for operating the pumping unit. The actuator 172-7 has a pivotable body having a yoke for linearly displacing an actuator mechanism when the body pivots under the influence of two shape memory alloy wires and a tension spring, such that alternative heating and cooling of the shape memory wires, reinforced by the action of the spring, pivots the body. However, the conventional infusion pump system 100E may require an inlet that admits fluid via a flexible tubing. This flexible tubing may be compressed in a coordinated manner to collect a small volume of fluid which is then dispensed via the outlet. The working principle may be similar to that of a peristaltic pump vs. the syringe pump principle.

Most of the conventional apparatus may use the SMA actuators or motors, however, the conventional apparatus may have restriction on the range of delivery resolutions, delivery capacity available, which may not be flexible to adapt the conventional apparatus to any fluid delivery application requiring any delivery resolution. Further, the aforementioned conventional devices/systems may or may not be reversible, in case of reversible scenario it may not be practically feasible, since each actuation during delivery may need to be precise. If the same movement resolution is maintained during reversal, then it can take significant time for the mechanism of conventional devices/system to completely reverse to an original starting point. Further, the conventional devices/system may also use a flexible suction cup to draw fluid from an inlet and releases it to the outlet. Once the reservoir is empty, the reservoir may need to be replaced. The delivery volume resolution of the device may be dependent on the design of the suction cup and the size. In case of conventional devices/system based on the syringe pump principle, the conventional devices/system may utilize a gear train to transmit motor rotation to the lead screw. This requires precise manufacturing tolerances for the gears as well as a high-resolution motor-encoder set.

Therefore, there is a need in the art to develop and design a scalable fluid delivery to achieve minimal motion of a syringe plunger to overcome or mitigate the above-mentioned and other limitations of the existing solutions and utilize techniques, which are robust, accurate, efficient, quick, and simple.

### OBJECTS OF THE PRESENT DISCLOSURE

Some of the objects of the present disclosure, which at least one embodiment herein satisfies are as listed herein below.

It is an object of the present disclosure to provide apparatus and method for scalable fluid delivery to achieve minimal motion of a syringe plunger using multiple kinematic chain elements/pathways actuated by a single Motion Actuating Device (MAD).

It is an object of the present disclosure to provide apparatus and method for scalable fluid delivery to achieve minimal/fine incremental motion in one direction and coarse, continuous motion in the reverse direction with the use of only a single Motion Actuating Device (MAD) as input.

It is an object of the present disclosure to provide apparatus for scalable fluid delivery with an incomplete teeth gear with feedback (i.e., encoder) used to achieve a similar level of micro-motion.

It is an object of the present disclosure to provide apparatus for scalable fluid delivery for discretization of input rotation to do away with the use of a precise micro-motor set.

It is an object of the present disclosure to provide apparatus for scalable fluid delivery, wherein the kinematic chain elements/pathways with a torque diode and clutches can also achieve a similar level of micro-motion.

It is an object of the present disclosure to provide apparatus for scalable fluid delivery for achieving the same level of micro-motion as required for infusion devices, which is performed in stages with each stage achieving a specific level of speed reduction, while allowing for quicker plunger motion during refill/change of a cartridge/barrel/fluid delivery tube.

It is an object of the present disclosure to provide apparatus for scalable fluid delivery for distributed speed reduction and the use of a single rotary actuator to achieve both linear, discrete micro-motion and continuous linear motion, to achieve the precision of movement required.

It is an object of the present disclosure to provide apparatus for scalable fluid delivery which reduces the overall device cost significantly, due to usage of simple kinematic elements.

It is an object of the present disclosure to provide apparatus for scalable fluid delivery with precise and repeatable motion as required by the infusion devices.

It is an object of the present disclosure to provide apparatus for scalable fluid delivery for different kinds of motion (fine, coarse, intermittent, continuous) within the same device via different kinematic pathways.

### SUMMARY

This section is provided to introduce certain objects and aspects of the present invention in a simplified form that are further described below in the detailed description. This summary is not intended to identify the key features or the scope of the claimed subject matter.

In an aspect, the present disclosure discloses a scalable fluid delivery apparatus. The scalable fluid delivery apparatus may include a housing to accommodate a syringe containing/to be filled with a fluid. The syringe comprises a barrel, a plunger within the barrel and a removable fluid delivery tube. Further, the scalable fluid delivery apparatus includes a driving unit removably coupled to the syringe, and operatively connected to a Motion Actuating Device (MAD) to advance the plunger of the syringe for expelling fluid from the barrel of the syringe. The driving unit includes a primary shaft coupled to the MAD, a secondary shaft, a wheel actuator comprising a wheel mounted on the secondary shaft. The wheel is a ratchet or an indexed wheel. The wheel actuator is at least one of a Compliant Ratchet Actuator (CRA), a Compliant Indexed Wheel Actuator (CIWA), a Rigid Linkages (RL) and a Rigid Actuator (RA). The driving unit further includes a plurality of clutches comprising a first clutch mounted on the primary shaft, a second clutch mounted on the secondary shaft, a third clutch mounted adjacent to the second clutch, and a fourth clutch mounted adjacent to the first clutch. Furthermore, the driving unit includes a threaded rotatable shaft rigidly coupled via a first end to the third clutch, and removably coupled via a second end to a Piston Drive Member (PDM). The PDM linearly advances the plunger of the syringe when in contact during a linear motion of the PDM based on a lead of the threaded rotatable shaft and rotation of the third clutch. Further, the driving unit includes a plurality of gears comprising a first gear rigidly coupled to the third clutch, a third gear coupled to the fourth clutch, a second gear coupled to the first gear and the third gear. Thereafter, the driving unit includes a switching mechanism coupled to the first clutch and the second clutch. The MAD transmits motion in a plurality of pathways corresponding to a plurality of resolutions. The plurality of pathways includes at least one of, a first kinematic pathway corresponding to a high linear resolution mode of the PDM and a second kinematic pathway corresponding to a low linear resolution mode of the PDM.

The first kinematic pathway corresponding to the high linear resolution mode of the PDM includes step of receiving an actuation from the switching mechanism to engage the second clutch and the third clutch, and to disengage the fourth clutch and the first clutch. Further, the first kinematic pathway includes step of performing an actuation by the MAD to transmit motion to the wheel actuator. The wheel actuator incrementally rotates the wheel, based on a type of coupling between the MAD and the wheel actuator. Furthermore, the first kinematic pathway includes step of rotating the engaged second clutch and the third clutch equivalent to an amount of rotation of the wheel. The third clutch and the threaded rotatable shaft rotate equivalent to an amount of rotation of the second clutch. Thereafter, the first kinematic pathway includes step of pushing the plunger of the syringe by a distance corresponding to a lead of the threaded rotatable shaft and an angle of rotation of the third clutch, to create a linear motion at the PDM. Also, the first kinematic pathway includes step of delivering the fluid comprised in the syringe, upon pushing the plunger. The second kinematic pathway corresponding to the low linear resolution mode of the PDM includes step of receiving an actuation from the switching mechanism to disengage the second clutch and the third clutch, and to engage the first clutch and the fourth clutch. Furthermore, the second kinematic pathway includes step of performing an actuation by the MAD to transmit a rotational motion to the wheel actuator. The wheel actuator incrementally rotates the wheel, based on a type of coupling between the MAD and the wheel actuator. Furthermore, the second kinematic pathway includes step of rotating the engaged fourth clutch and the first clutch equivalent to an amount of motion of the MAD. Thereafter, the second kinematic pathway includes step of transmitting rotation of the fourth clutch to the third clutch. The first gear, the third clutch and the threaded rotatable shaft rotates based on rotation transmitted from the second gear and the third gear. The third clutch and the threaded rotatable shaft rotates equivalent to an amount of a gear ratio of the third gear and the first gear or to an amount of an arbitrary ratio based on a gear train configuration. Furthermore, the second kinematic pathway includes step of performing forward motion or reverse motion of the PDM, corresponding to a lead and an angle of rotation of the third clutch and the threaded rotatable shaft. The forward motion, or the reverse motion of the PDM is for priming the barrel and the removable fluid delivery tube or to revert the PDM to an initial position, respectively, or to deliver the fluid.

In another aspect, the present disclosure discloses a method for scalable fluid delivery. The method includes a first kinematic pathway steps corresponding to a high linear resolution mode of a Piston Drive Member (PDM). The method for first kinematic pathway step includes receiving an actuation from a switching mechanism to engage a second clutch and a third clutch, and to disengage a fourth clutch and a first clutch. The method for first kinematic pathway step includes performing an actuation by a Motion Actuating Device (MAD) to transmit motion to a wheel actuator. The wheel actuator incrementally rotates a wheel, based on a type of coupling between the MAD and the wheel actuator. Furthermore, the method for first kinematic pathway step includes rotating the engaged second clutch and the third clutch equivalent to an amount of rotation of the wheel. The third clutch and a threaded rotatable shaft rotate equivalent to an amount of rotation of the second clutch. Furthermore, the method for first kinematic pathway step includes pushing a plunger of a syringe by a distance corresponding to a lead of the threaded rotatable shaft and an angle of rotation of the third clutch, to create a linear motion at the Piston Drive Member (PDM). Thereafter, the method for first kinematic pathway step includes delivering a fluid comprised in the syringe, upon pushing the plunger. The method includes a second kinematic pathway steps corresponding to a low linear resolution mode of a Piston Drive Member (PDM). The method of the second kinematic pathway step includes receiving an actuation from the switching mechanism to disengage the second clutch and the third clutch, and to engage the first clutch and the fourth clutch. Furthermore, the method of the second kinematic pathway step includes performing an actuation by the MAD to transmit a rotational motion to the wheel actuator. The wheel actuator incrementally rotates the wheel, based on a type of coupling between the MAD and the wheel actuator. Furthermore, the method of the second kinematic pathway step includes rotating the engaged fourth clutch and the first clutch equivalent to an amount of motion of the MAD. Thereafter, the method of the second kinematic pathway step includes transmitting rotation of the fourth clutch to the third clutch, wherein the first gear, the third clutch and the threaded rotatable shaft rotates based on rotation transmitted from the second gear and the third gear. The third clutch and the threaded rotatable shaft rotates equivalent to an amount of a gear ratio of the third gear and the first gear or to an amount of an arbitrary ratio based on a gear train configuration. Further, the method of the second kinematic pathway step includes performing forward motion, or reverse motion of the PDM, corresponding to a lead and an angle of rotation of the third clutch and the threaded rotatable shaft. The forward motion, or reverse motion of the PDM is for priming the barrel and a removable fluid delivery tube or to revert the PDM to an initial position, respectively, or to deliver the fluid.

In yet another aspect, the present disclosure discloses a scalable fluid delivery apparatus. The scalable fluid delivery apparatus includes a first kinematic pathway corresponding to a high linear resolution mode of a Piston Drive Member (PDM). The first kinematic pathway includes receiving an actuation from a switching mechanism to engage a second clutch and a third clutch, and to disengage a fourth clutch and a first clutch and performing an actuation by a motor to transmit a rotational motion to a Compliant Ratchet Actuator (CRA) via an Eccentric Cam (EC) attached to a primary shaft. The CRA incrementally rotates the ratchet wheel, based on an eccentricity of the EC. Furthermore, the first kinematic pathway includes rotating the engaged second clutch and the third clutch, equivalent to an amount of rotation of the ratchet wheel. The third clutch and a threaded rotatable shaft rotate equivalent to an amount of rotation of the second clutch. Furthermore, the first kinematic pathway includes pushing a plunger of a syringe by a distance corresponding to a lead of the threaded rotatable shaft and angle of rotation of the third clutch, to create a linear motion at the PDM. Thereafter, the first kinematic pathway includes delivering a fluid comprised in the syringe, upon pushing the plunger. The scalable fluid delivery apparatus includes a second kinematic pathway corresponding to the low linear resolution mode of the PDM. The second kinematic pathway includes receiving an actuation from the switching mechanism to disengage the second clutch and the third clutch, and to engage the first clutch and the fourth clutch. Further, the second kinematic pathway includes performing an actuation by the motor to transmit a rotational motion to a Compliant Ratchet Actuator (CRA) via the EC attached to the primary shaft. The CRA incrementally rotates the ratchet wheel, based on the eccentricity of the EC. Furthermore, the second kinematic pathway includes rotating the engaged fourth clutch and the first clutch equivalent to an amount of rotation of the motor. Thereafter, the second kinematic pathway includes transmitting rotation of the fourth clutch to the third clutch. The first gear, the third clutch and the threaded rotatable shaft rotates based on rotation transmitted from the second gear and the third gear. The third clutch and the threaded rotatable shaft rotates equivalent to an amount of a gear ratio of the third gear and the first gear or to an amount of an arbitrary ratio based on a gear train configuration. Further, the second kinematic pathway includes performing forward or reverse motion of the PDM, corresponding to a lead and an angle of rotation of the third clutch and the threaded rotatable shaft. The forward motion, or the reverse motion of the PDM is for priming a barrel of a syringe and a removable fluid delivery tube or to revert the PDM to an initial position, respectively, or to deliver the fluid.

In yet another aspect, the present disclosure discloses a scalable fluid delivery apparatus comprising at least one of a one-way bearing or a clutch bearing within the primary shaft to restrict the motion transmitted to the wheel actuator.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The accompanying drawings, which are incorporated herein, and constitute a part of this invention, illustrate exemplary embodiments of the disclosed methods and systems in which like reference numerals refer to the same parts throughout the different drawings. Components in the drawings are not necessarily to scale, emphasis instead being placed upon clearly illustrating the principles of the present invention. Some drawings may indicate the components using block diagrams and may not represent the internal circuitry/subcomponents of each component. It will be appreciated by those skilled in the art that invention of such drawings includes the invention of electrical components, electronic components or circuitry commonly used to implement such components.

FIGS. 1A-1E illustrate a perspective view of conventional apparatus for fluid delivery, such as a shape memory actuated dispensing pump, a packaged peristaltic micropump, a compact medical pump device, a drug delivery pump drive, and an infusion pump system, respectively.

FIG. 2A illustrates a perspective view of an exemplary scalable fluid delivery apparatus of the present disclosure, in accordance with an embodiment of the present disclosure.

FIG. 2B illustrates a perspective view of an exemplary implementation of a scalable fluid delivery apparatus, in accordance with an embodiment of the present disclosure.

FIG. 3 illustrates a flow diagram representation depicting a method for scalable fluid delivery, in accordance with an embodiment of the present disclosure.

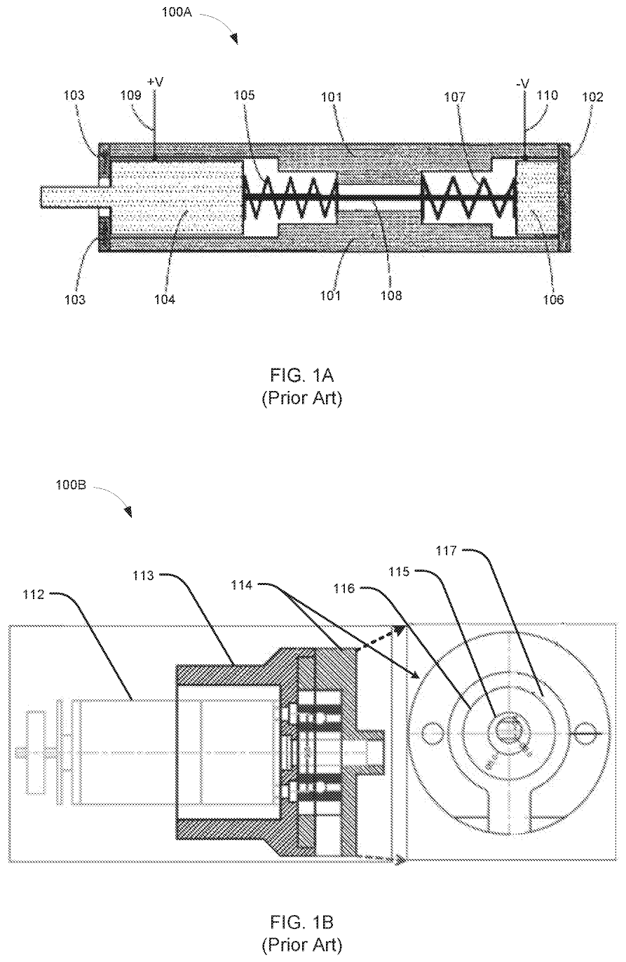

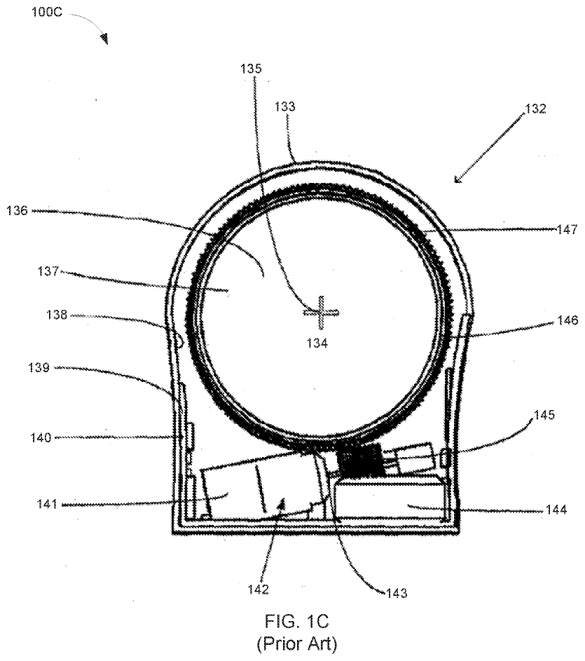

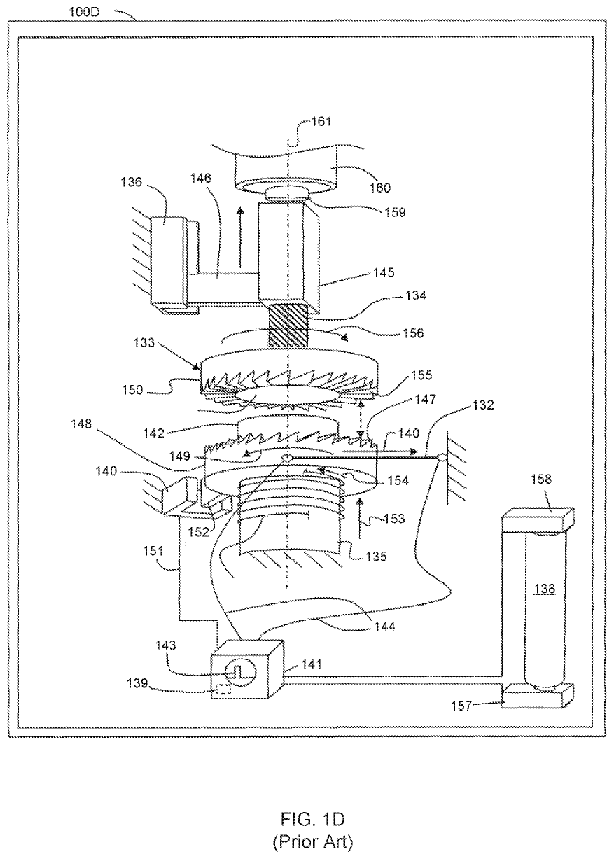

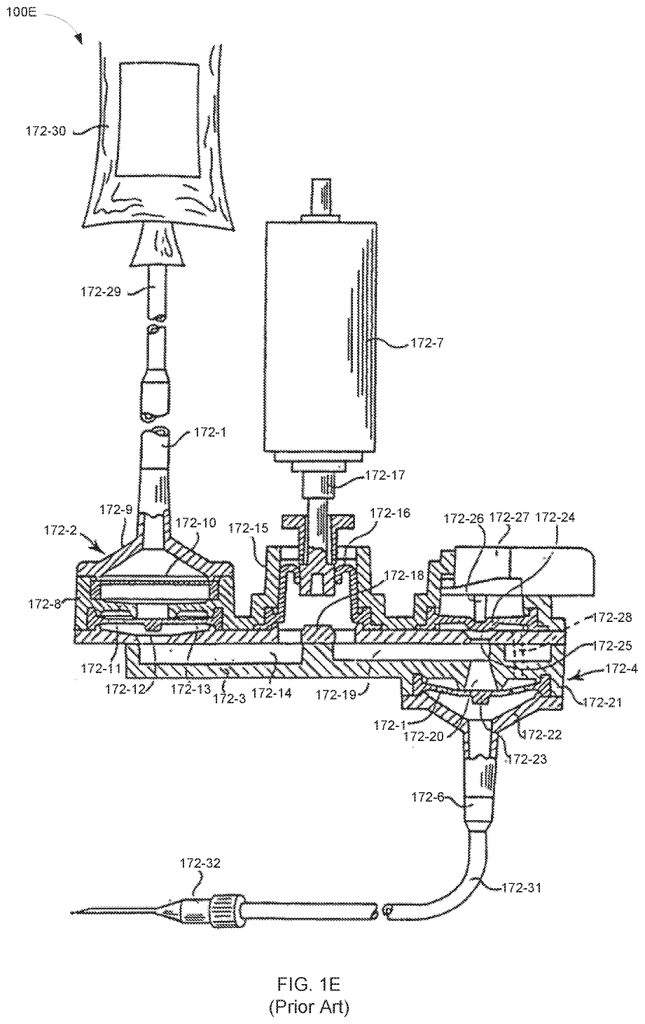

The foregoing shall be more apparent from the following more detailed description of the invention.

### DETAILED DESCRIPTION OF INVENTION

In the following description, for the purposes of explanation, various specific details are set forth in order to provide a thorough understanding of embodiments of the present disclosure. It will be apparent, however, that embodiments of the present disclosure may be practiced without these specific details. Several features described hereafter can each be used independently of one another or with any combination of other features. An individual feature may not address all of the problems discussed above or might address only some of the problems discussed above. Some of the problems discussed above might not be fully addressed by any of the features described herein.

The ensuing description provides exemplary embodiments only, and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the spirit and scope of the invention as set forth.

Specific details are given in the following description to provide a thorough understanding of the embodiments. However, it will be understood by one of ordinary skill in the art that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination can correspond to a return of the function to the calling function or the main function.

The word “exemplary” and/or “demonstrative” is used herein to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive—in a manner similar to the term “comprising” as an open transition word—without precluding any additional or other elements.

Reference throughout this specification to “one embodiment” or “an embodiment” or “an instance” or “one instance” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Embodiments of the present disclosure provides apparatus and method for scalable fluid delivery to achieve minimal motion of a syringe plunger using multiple kinematic chain elements/pathways actuated by a single Motion Actuating Device (MAD). The present disclosure provides apparatus and method for scalable fluid delivery to achieve minimal/fine incremental motion in one direction and coarse, continuous motion in the reverse direction with the use of only a single Motion Actuating Device (MAD) as input. The present disclosure provides apparatus for scalable fluid delivery with an incomplete teeth gear with feedback (i.e., encoder) used to achieve a similar level of micro-motion. The present disclosure provides apparatus for scalable fluid delivery for discretization of input rotation to do away with the use of a precise micro-motor set. The present disclosure provides apparatus for scalable fluid delivery wherein the kinematic chain elements/pathways with a torque diode and clutches can also achieve a similar level of micro-motion.

Embodiments of the present disclosure provides apparatus for scalable fluid delivery for achieving the same level of micro-motion as required for infusion devices, which is performed in stages with each stage achieving a specific level of speed reduction, while allowing for quicker plunger motion during refill/change of a cartridge/barrel/fluid delivery tube. The present disclosure provides apparatus for scalable fluid delivery for distributed speed reduction and the use of a single rotary actuator to achieve both linear, discrete micro-motion and continuous linear motion, to achieve the precision of movement required. The present disclosure provides apparatus for scalable fluid delivery which reduces the overall device cost significantly, due to usage of simple kinematic elements. The present disclosure provides apparatus for scalable fluid delivery with precise and repeatable motion as required by the infusion devices. The present disclosure provides apparatus for scalable fluid delivery for different kinds of motion (fine, coarse, intermittent, continuous) within the same device via different kinematic pathways.

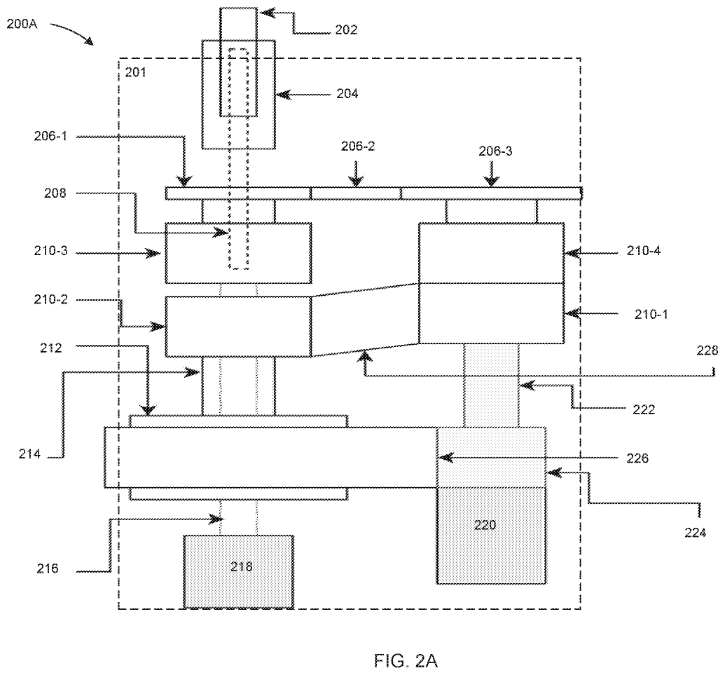

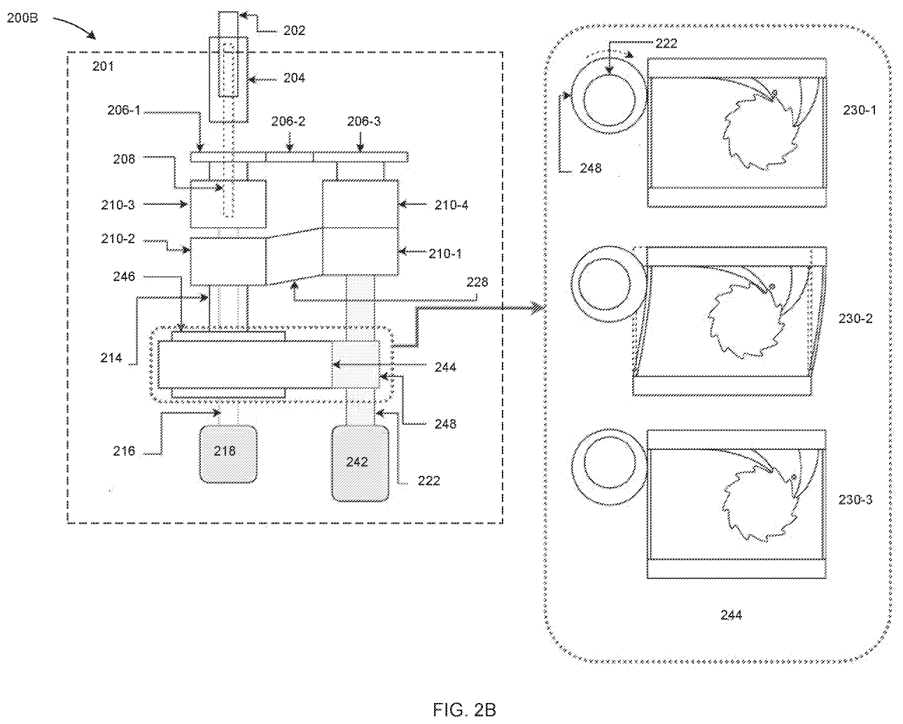

Referring to FIG. 2A, illustrating a perspective view implementing a scalable fluid delivery apparatus 200A, in accordance with an embodiment of the present disclosure. In an embodiment, the scalable fluid delivery apparatus 200A includes a housing (not shown in FIG. 2A) and a driving unit 201 (shown in FIG. 2A). The housing may accommodate a syringe (not shown in FIG. 2A) containing a fluid/liquid. The types of fluids/liquids that can be delivered by the scalable fluid delivery apparatus 200A may include, but are not limited to, insulin, antibiotics, nutritional fluids, Total Parenteral Nutrition (TPN), analgesics, morphine, hormones or hormonal drugs, gene therapy drugs, anticoagulants, analgesics, cardiovascular medications, Azidothymidine (AZT) or chemotherapeutics, medications, drugs, vitamins, vaccines, peptides, or the like. However, it will be recognized that further embodiments of the present disclosure may be used in other devices that require compact and accurate drive mechanisms. In addition, other shape altering materials, such as piezo-electric materials, or the like, may be used. The types of medical conditions that the scalable fluid delivery apparatus 200A may be used to treat includes, but are not limited to, diabetes, cardiovascular disease, pain, chronic pain, cancer, Acquired Immune Deficiency Syndrome (AIDS), neurological diseases, Alzheimer's Disease, Amyotrophic Lateral Sclerosis (ALS), Hepatitis, Parkinson's disease, or spasticity, and the like.

The syringe includes, but are not limited to, a barrel, and a plunger within the barrel, a removable fluid delivery tube (not shown in FIG. 2A), and the like. In one implementation, the fluid delivery tube may be retained, and in another implementation the fluid delivery tube may be removed. The scalable fluid delivery apparatus 200A including the driving unit 201 may be removably coupled to the syringe. The driving unit 201 may include a Motion Actuating Device (MAD) 220, to advance the plunger of the syringe for expelling fluid from the barrel of the syringe. In an embodiment, the MAD 220 may transmit motion in a plurality of pathways corresponding to a plurality of resolutions. The driving unit 201 including the MAD 220 may be coupled to a primary shaft 222. The driving unit 201 may also include a secondary shaft 214. Further, the driving unit 201 includes a wheel actuator 226, and wheel actuator 226 includes a wheel 212 mounted on the secondary shaft 214. The wheel 212 may include, but are not limited to a rachet, an indexed wheel, and the like. Further, the wheel actuator 226 may include, but are not limited to, a Compliant Ratchet Actuator (CRA), a Compliant Indexed Wheel Actuator (CIWA), Rigid Linkages (RL), a Rigid Actuator (RA), and the like. The wheel actuator 226 may be coupled to the primary shaft 222 using any suitable coupling mechanism 224. In an embodiment, the driving unit 201 may include a plurality of clutches. The plurality of clutches may include a first clutch 210-1 mounted on the primary shaft 222, a second clutch 210-2 mounted on the secondary shaft 214, a third clutch 210-3 mounted adjacent to the second clutch 210-2, and a fourth clutch 210-4 mounted adjacent to the first clutch 210-1. In an instance, the plurality of clutches may be coupled to each other using a Hirth coupling or Hirth joint, which may be a type of mechanical connection and used to connect two pieces of a shaft together via tapered teeth that mesh on the end faces of each half shaft. Further, the driving unit 201 may include a threaded rotatable shaft 208. The threaded rotatable shaft 208 may be rigidly coupled via a first end to the third clutch 210-3, and removably coupled via a second end to a Piston Drive Member (PDM) 202. In an embodiment, the PDM 202 may linearly advance the plunger of the syringe when in contact, and during a linear motion of the PDM 202 based on a lead of the threaded rotatable shaft 208 and rotation of the third clutch 210-3. The threaded rotatable shaft 208 may be used as a linkage, to translate rotary motion into linear motion. The plurality of pathways includes, but are not limited to, a first kinematic pathway corresponding to a high linear resolution mode of the PDM 202, a second kinematic pathway corresponding to a low linear resolution mode of the PDM 202, and the like. In an embodiment, the first kinematic pathway corresponding to the high linear resolution mode of the PDM 202 includes one or more steps. Thereafter, the driving unit 201 may include a plurality of gears. The plurality of gears may include a first gear 206-1 rigidly coupled to the third clutch 210-3, a third gear 206-3 coupled to the fourth clutch 210-4, a second gear 206-2 coupled to the first gear 206-1 and the third gear 206-3. Further, the driving unit 201 may include a switching mechanism 228 coupled to the first clutch 210-1 and the second clutch 210-2. The first kinematic pathway may include receiving an actuation from the switching mechanism 228 to engage the second clutch 210-2 and the third clutch 210-3, and to disengage the fourth clutch 210-4 and the first clutch 210-1. Further, the first kinematic pathway may include performing an actuation by the MAD 220 to transmit motion to the wheel actuator 226. The MAD 220 may rotate to provide intermittent motion or continuous motion using at least one of the first kinematic pathway and the second kinematic pathway. The MAD 220 and switching mechanism 228 may include, but are not limited to, a motor, a linear motor, a liner actuator, a Shape Memory Alloy (SMA) actuator, a Solenoid, and the like. The SMA may be a memory alloy that can be deformed when cold but returns to its pre-deformed (“remembered”) shape when heated. The SMA may also be called memory metal, memory alloy, smart metal, smart alloy, or muscle wire. Parts made of SMA can be lightweight, solid-state alternatives to conventional actuators such as hydraulic, pneumatic, and motor-based systems. The wheel actuator 226 may incrementally rotate (not shown in FIG. 2A) the wheel 212, based on a type of coupling 224 between the MAD 220 and the wheel actuator 226. For instance, the wheel actuator 226 may include a primary shaft 222 and an Eccentric Cam (EC) (not shown in FIG. 2A) in contact with the wheel actuator 226, which is in contact with the wheel 212. The wheel actuator 226 may be actuated using, but not limited to, an Eccentric Cam (EC) coupled to a motor, a linear motor, a linear actuator, a Shape Memory Alloy (SMA) actuator, a Solenoid, and the like. The type of coupling between the MAD 220 and the wheel actuator 226 includes an Eccentric Cam (EC), and the like. Upon wheel actuator 226 incrementally rotating the wheel 212, based on the type of coupling between the MAD 220 and the wheel actuator 226, rotating the first clutch 210-1 corresponding to an amount of motion by the MAD 220. The rotation of the first clutch 210-1 may not be transmitted to the fourth clutch 210-4, due to disengagement between the first clutch 210-1 and the fourth clutch 210-4.

In some implementations, the scalable fluid delivery apparatus 200A may also include a processor (not shown in FIG. 2A) or electronic microcontroller (not shown in FIG. 2A) connected to the MAD 220. The processor may be programmed to cause a flow of fluid based on flow instructions from a separate, remote-control device (not shown in FIG. 2A). In some implementations, the scalable fluid delivery apparatus 200A may also further include a wireless receiver connected to the processor for receiving the flow instructions from the separate, remote-control device and delivering the flow instructions to the processor.

In an embodiment, the first kinematic pathway may include rotating the engaged second clutch 210-2 and the third clutch 210-3 equivalent to an amount of rotation (not shown in FIG. 2A) of the wheel 212. Upon rotation of the second clutch 210-2 and the third clutch 210-3, the third gear 206-3 and the fourth clutch 210-4 may subsequently rotate via the second gear 206-2. The rotation of the fourth clutch 210-4 may not be transmitted to the first clutch 210-1, due to disengagement between the fourth clutch 210-4 and the first clutch 210-1. The third clutch 210-3 and the threaded rotatable shaft 208 may rotate equivalent to an amount of rotation of the second clutch 210-2. Thereafter, the first kinematic pathway may include pushing the plunger of the syringe by a distance corresponding to a lead of the threaded rotatable shaft 208 and an angle of rotation of the third clutch 210-3, to create a linear motion at the PDM 202. Further, the first kinematic pathway may include delivering the fluid within the syringe, upon pushing the plunger. For insulin applications at a concentration of, for example, 100 units per microliter (units/ml), or the like, a pulse volume of less than two microliters, and typically a half of a microliter, is appropriate.

In an embodiment, the second kinematic pathway corresponding to the low linear resolution mode of the PDM 202 may include one or more steps. The second kinematic pathway may include receiving an actuation from the switching mechanism 228 to disengage the second clutch 210-2 and the third clutch 210-3, and to engage the first clutch 210-1 and the fourth clutch 210-4. The second kinematic pathway may include performing an actuation by the MAD 220 to transmit a rotational motion to the wheel actuator 226. The wheel actuator 226 may incrementally rotate the wheel 212, based on a type of coupling between the MAD 220 and the wheel actuator 226. Upon, the wheel actuator 226 incrementally rotating the wheel 212, based on the type of coupling between the MAD 220 and the wheel actuator 226, rotating the second clutch 210-2 equivalent to an amount of rotation by the wheel 212. The rotation of the second clutch 210-2 may not be transmitted to the third clutch 210-3, due to disengagement between the second clutch 210-2 and the third clutch 210-3.

Furthermore, the second kinematic pathway may include rotating the engaged fourth clutch 210-4 and the first clutch 210-1 equivalent to an amount of motion of the MAD 220. Furthermore, the second kinematic pathway may include transmitting rotation of the fourth clutch 210-4 to the third clutch 210-3. The first gear 206-1, the third clutch 210-3 and the threaded rotatable shaft 208 may rotate based on rotation transmitted from the second gear 206-2 and the third gear 206-3. The third clutch 210-3 and the threaded rotatable shaft 208 may rotate equivalent to an amount of a gear ratio of the third gear 206-3 and the first gear 206-1 or to an amount of an arbitrary ratio based on a gear train configuration. Upon rotation of the third gear 206-3 and the fourth clutch 210-4, the third clutch 210-3, the first gear 206-1 and the threaded rotatable shaft 208 may subsequently rotate via the second gear 206-2. The rotation of the third clutch 210-3 may not be transmitted to the second clutch 210-2, due to disengagement between the third clutch 210-3 and the second clutch 210-2. Thereafter, the second kinematic pathway may include performing forward motion or reverse motion of the PDM 202, corresponding to a lead and an angle of rotation of the third clutch 210-3 and the threaded rotatable shaft 208. The forward motion, or the reverse motion of the PDM 202 may be for priming the barrel and the removable fluid delivery tube or to revert the PDM 202 to an initial position, respectively, or to deliver fluid. The PDM 202 may be constrained by a sliding joint 204 with a non-circular cross-section. The non-circular cross-section may include, but are not limited to, a square, a rectangle, a triangle, a parallelogram, a rhombus, a trapezium, a quadrilateral, an oval, a diagonal polygon, a hexagonal, a polygonal, and the like. The non-circular cross-section of the sliding joint 204 may not allow the sliding joint 204 to rotate upon rotation of the threaded rotatable shaft 208, or the third clutch 210-3, and only moves linearly. The third clutch 210-3 may be coupled to an encoder 218 via a shaft 216 independent of the secondary shaft 214. The encoder 218 may enable confirmation or control of the amount of rotation of the third clutch 210-3 or the threaded rotatable shaft 208.

In one implementation, a scalable fluid delivery apparatus 200B may include a first kinematic pathway corresponding to a high-resolution mode of a Piston Drive Member (PDM) 202. The first kinematic pathway may include receiving an actuation from the switching mechanism 228 to engage a second clutch 210-2 and a third clutch 210-3, and to disengage a fourth clutch 210-4 and a first clutch 210-1, as shown in FIG. 2B. Further, the first kinematic pathway includes performing an actuation by a motor 242 (such as the MAD 220) as shown in FIG. 2B, to transmit a rotational motion to a Compliant Ratchet Actuator (CRA) 244 as shown in FIG. 2B, via an Eccentric Cam (EC) 248 as shown in FIG. 2B, attached to a primary shaft 222. The enlarged cross section of the CRA 244 and working is shown in right side of FIG. 2B. The CRA 244 may incrementally rotate a ratchet wheel 246, based on a type of coupling 224 between the motor 242 and the CRA 244, as shown in FIG. 2B. For instance, the cross-section of the CRA 244 as shown in FIG. 2B may include the primary shaft 222 and the Eccentric Cam (EC) 248 in contact with the CRA 244, which is in contact with the ratchet wheel 246. The EC 248 may be a part of the primary shaft 222. The CRA 244 may incrementally rotate the ratchet wheel 246 as shown in FIG. 2B (as the wheel 212), based on an eccentricity of the EC 248, depicted in steps 230-1, 230-2 and 230-3 as shown in FIG. 2B. The first kinematic pathway may include rotating the engaged second clutch 210-2 and the third clutch 210-3, equivalent to an amount of rotation of the ratchet wheel 246. The third clutch 210-3 and a threaded rotatable shaft 208 may rotate equivalent to an amount of rotation of the second clutch 210-2. Furthermore, the first kinematic pathway may include pushing a plunger of a syringe by a distance corresponding to a lead of the threaded rotatable shaft 208 and angle of rotation of the third clutch 210-3, to create a linear motion at the PDM 202. The first kinematic pathway may include delivering a fluid in the syringe, upon pushing the plunger. In another implementation, the second kinematic pathway corresponding to a low linear resolution mode of the PDM 202 may include receiving an actuation from the switching mechanism 228 to disengage the second clutch 210-2 and the third clutch 210-3, and to engage the first clutch 210-1 and the fourth clutch 210-4. Further, the second kinematic pathway may include performing an actuation by the motor 242 as the MAD 220 to transmit a rotational motion to the CRA 244 such as the wheel actuator 226 via the EC 248 attached to the primary shaft 222. The CRA 244 may incrementally rotate the ratchet wheel 246 such as the wheel 212, based on the eccentricity of the EC 248. Further, the second kinematic pathway may include rotating the engaged fourth clutch 210-4 and the first clutch 210-1 equivalent to an amount of rotation of the motor 242. Furthermore, the second kinematic pathway may include transmitting rotation of the fourth clutch 210-4 to the third clutch 210-3. The first gear 206-1, the third clutch 210-3 and the threaded rotatable shaft 208 may rotate based on rotation transmitted from the second gear 206-2 and the third gear 206-3. The third clutch 210-3 and the threaded rotatable shaft 208 may rotate equivalent to an amount of a gear ratio of the third gear 206-3 and the first gear 206-1 or to an amount of an arbitrary ratio based on a gear train configuration. Thereafter, the second kinematic pathway may include performing forward or reverse motion of the PDM 202, corresponding to a lead and an angle of rotation of the third clutch 210-3 and the threaded rotatable shaft 208. The forward motion, or the reverse motion of the PDM 202 may be for priming a barrel of a syringe and a removable fluid delivery tube or to revert the PDM 202 to an initial position, respectively, or to deliver fluid.

In yet another implementation, the scalable fluid delivery apparatus 200A or the scalable fluid delivery apparatus 200B may further include one-way bearing or a clutch bearing within the primary shaft 222 to restrict the motion transmitted to the wheel actuator 226.

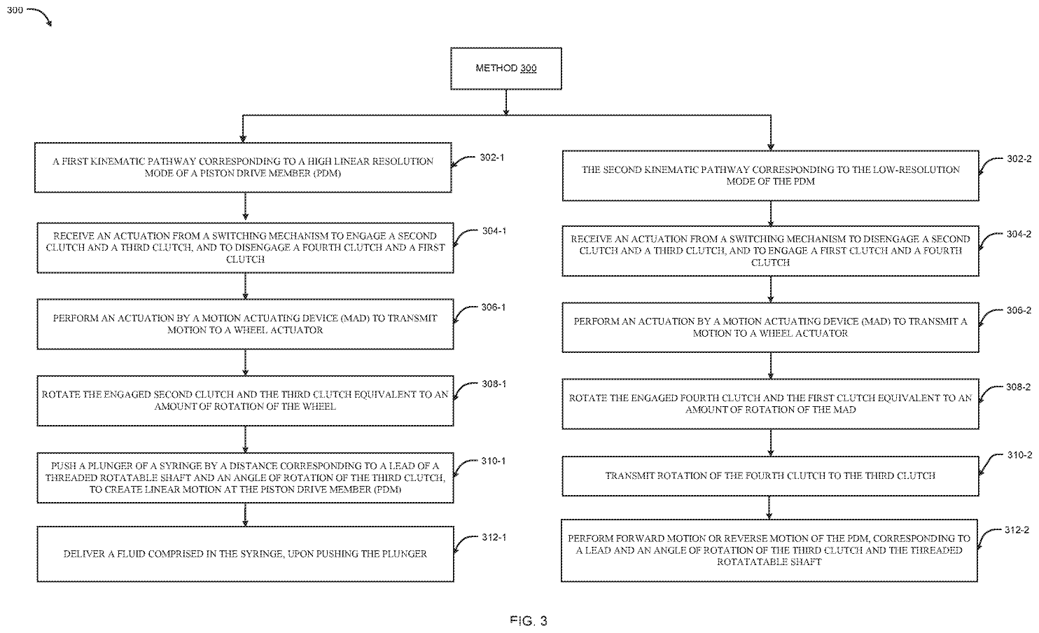

FIG. 3 illustrates a flow diagram representation depicting a method 300 for scalable fluid delivery, in accordance with an embodiment of the present disclosure.

The method 300 may include first kinematic pathway steps and second kinematic pathway steps. At block 302-1, the method 300 includes a first kinematic pathway corresponding to a high linear resolution mode of the PDM 202. At block 304-1, the method 300 includes receiving, by the scalable fluid delivery apparatus 200A, an actuation from the switching mechanism 228, to engage a second clutch 210-2 and the third clutch 210-3, and to disengage the fourth clutch 210-4 and the first clutch 210-1. At block 306-1, the method 300 includes performing, by the scalable fluid delivery apparatus 200A, an actuation by the Motion Actuating Device (MAD) 220 to transmit motion to the wheel actuator 226. The wheel actuator 226 may incrementally rotate the wheel 212, based on the type of coupling 224 between the MAD 220 and the wheel actuator 226. At block 308-1, the method 300 includes rotating, by the scalable fluid delivery apparatus 200A, the engaged second clutch 210-2 and the third clutch 210-3 equivalent to an amount of rotation of the wheel 212. The third clutch 210-3 and the threaded rotatable shaft 208 may rotate equivalent to an amount of rotation of the second clutch 210-2. At block 310-1 the method 300 includes pushing, by the scalable fluid delivery apparatus 200A, a plunger of a syringe by a distance corresponding to a lead of the threaded rotatable shaft 208 and an angle of rotation of the third clutch 210-3, to create a linear motion at the Piston Drive Member (PDM) 202. At block 312-1, the method 300 includes delivering, by the scalable fluid delivery apparatus 200A, a fluid comprised in the syringe, upon pushing the plunger.

At block 302-2, the method 300 includes the second kinematic pathway corresponding to the low linear resolution mode of the PDM 202. At block 304-2, the method 300 includes receiving, by the scalable fluid delivery apparatus 200A, from the switching mechanism 228 to disengage the second clutch 210-2 and the third clutch 210-3, and to engage the first clutch 210-1 and the fourth clutch 210-4. At block 306-2 the method 300 includes performing, by the scalable fluid delivery apparatus 200A, an actuation by the MAD 220 to transmit motion to the wheel actuator 226. The wheel actuator 226 may incrementally rotate the wheel 212, based on a type of coupling 224 between the MAD 220 and the wheel actuator 226. At block 308-2, the method 300 includes rotating, by the scalable fluid delivery apparatus 200A, the engaged fourth clutch 210-4 and the first clutch 210-1 equivalent to an amount of motion of the MAD 220. At block 310-2 the method 300 includes transmitting, by the scalable fluid delivery apparatus 200A, rotation of the fourth clutch 210-4 to the third clutch 210-3. The first gear 206-1, the third clutch 210-3 and the threaded rotatable shaft 208 may rotate based on rotation transmitted from the second gear 206-2 and the third gear 206-3. The third clutch 210-3 and the threaded rotatable shaft 208 may rotate equivalent to an amount of a gear ratio of the third gear 206-3 and the first gear 206-1 or to an amount of an arbitrary ratio based on a gear train configuration. At block 312-2, the method 300 includes performing, by the scalable fluid delivery apparatus 200A, forward motion, or reverse motion of the PDM 202, corresponding to a lead and an angle of rotation of the third clutch 210-3 and the threaded rotatable shaft 208. The forward motion, or the reverse motion of the PDM 202 may be for priming the barrel and a removable fluid delivery tube or to revert the PDM 202 to an initial position, respectively, or to deliver fluid.

While considerable emphasis has been placed herein on the preferred embodiments, it will be appreciated that many embodiments can be made and that many changes can be made in the preferred embodiments without departing from the principles of the invention. These and other changes in the preferred embodiments of the invention will be apparent to those skilled in the art from the disclosure herein, whereby it is to be distinctly understood that the foregoing descriptive matter to be implemented merely as illustrative of the invention and not as limitation.

### Advantages of the Present Disclosure

The present disclosure provides apparatus and method for scalable fluid delivery to achieve minimal motion of a syringe plunger using multiple kinematic chain elements/pathways actuated by a single Motion Actuating Device (MAD).

The present disclosure provides apparatus and method for scalable fluid delivery to achieve minimal/fine, incremental motion in one direction and coarse, continuous motion in the reverse direction with the use of only a single Motion Actuating Device (MAD) as input.

The present disclosure provides apparatus for scalable fluid delivery with an incomplete teeth gear with feedback (i.e., encoder) used to achieve a similar level of micro-motion.

The present disclosure provides apparatus for scalable fluid delivery for discretization of input rotation to do away with the use of a precise micro-motor set.

The present disclosure provides apparatus for scalable fluid delivery, wherein the kinematic chain elements/pathways with a torque diode and clutches can also achieve a similar level of micro-motion.

The present disclosure provides apparatus for scalable fluid delivery for achieving the same level of micro-motion as required for infusion devices, which is performed in stages with each stage achieving a specific level of speed reduction, while allowing for quicker plunger motion during refill/change of a cartridge/barrel/fluid delivery tube.

The present disclosure provides apparatus for scalable fluid delivery for distributed speed reduction and the use of a single rotary actuator to achieve both linear, discrete micro-motion and continuous, linear motion, to achieve the precision of movement required.

The present disclosure provides apparatus for scalable fluid delivery which reduce the overall device cost significantly, due to usage of simple kinematic elements.

The present disclosure provides apparatus for scalable fluid delivery with precise and repeatable motion as required by the infusion devices.

The present disclosure provides apparatus for scalable fluid delivery for different kinds of motion (fine, coarse, intermittent, continuous) within the same device via different kinematic pathways.

1. A scalable fluid delivery apparatus comprising a housing to accommodate a syringe containing/to be filled with a fluid, wherein the syringe comprises a barrel, a plunger within the barrel and a removable fluid delivery tube, the scalable fluid delivery apparatus comprising:
a driving unit removably coupled to the syringe, and operatively connected to a Motion Actuating Device (MAD) to advance the plunger of the syringe for expelling fluid from the barrel of the syringe, wherein the driving unit comprising:
a primary shaft coupled to the MAD;
a secondary shaft;
a wheel actuator comprising a wheel mounted on the secondary shaft, wherein the wheel is a ratchet or an indexed wheel, wherein the wheel actuator is at least one of a Compliant Ratchet Actuator (CRA), a Compliant Indexed Wheel Actuator (CIWA), a Rigid Linkages (RL) and a Rigid Actuator (RA);
a plurality of clutches comprising a first clutch mounted on the primary shaft, a second clutch mounted on the secondary shaft, a third clutch mounted adjacent to the second clutch, and a fourth clutch mounted adjacent to the first clutch;
a threaded rotatable shaft rigidly coupled via a first end to the third clutch, and removably coupled via a second end to a Piston Drive Member (PDM), wherein the PDM linearly advances the plunger of the syringe when in contact, and during a linear motion of the PDM based on a lead of the threaded rotatable shaft and rotation of the third clutch;
a plurality of gears comprising a first gear rigidly coupled to the third clutch, a third gear coupled to the fourth clutch, a second gear coupled to the first gear and the third gear;
a switching mechanism coupled to the first clutch and the second clutch;
wherein the MAD is configured to transmit motion in a plurality of pathways corresponding to a plurality of resolutions, wherein the plurality of pathways comprises at least one of a first kinematic pathway corresponding to a high linear resolution mode of the PDM and a second kinematic pathway corresponding to a low linear resolution mode of the PDM,
the first kinematic pathway corresponding to the high linear resolution mode of the PDM comprising steps of:
receiving an actuation from the switching mechanism to engage the second clutch and the third clutch, and to disengage the fourth clutch and the first clutch;
performing an actuation by the MAD to transmit motion to the wheel actuator, wherein the wheel actuator incrementally rotates the wheel, based on a type of coupling between the MAD and the wheel actuator;
rotating the engaged second clutch and the third clutch equivalent to an amount of rotation of the wheel, wherein the third clutch and the threaded rotatable shaft rotate equivalent to an amount of rotation of the second clutch;
pushing the plunger of the syringe by a distance corresponding to a lead of the threaded rotatable shaft and an angle of rotation of the third clutch, to create a linear motion at the PDM; and
delivering the fluid comprised in the syringe, upon pushing the plunger; and
the second kinematic pathway corresponding to the low linear resolution mode of the PDM comprising steps of:
receiving an actuation from the switching mechanism to disengage the second clutch and the third clutch, and to engage the first clutch and the fourth clutch;
performing an actuation by the MAD to transmit motion to the wheel actuator, wherein the wheel actuator incrementally rotates the wheel, based on a type of coupling between the MAD and the wheel actuator;
rotating the engaged fourth clutch and the first clutch equivalent to an amount of motion of the MAD;
transmitting rotation of the fourth clutch to the third clutch, wherein the first gear, the third clutch and the threaded rotatable shaft rotates based on rotation transmitted from the second gear and the third gear, wherein the third clutch and the threaded rotatable shaft rotates equivalent to an amount of a gear ratio of the third gear and the first gear or to an amount of an arbitrary ratio based on a gear train configuration; and
performing forward motion or reverse motion of the PDM, corresponding to a lead and an angle of rotation of the third clutch and the threaded rotatable shaft, wherein the forward motion, or the reverse motion of the PDM is for priming the barrel and the removable fluid delivery tube or to revert the PDM to an initial position, respectively, or to deliver the fluid.

2. The scalable fluid delivery apparatus as claimed in claim 1, wherein, in the first kinematic pathway corresponding to the high linear resolution mode of the PDM, the wheel actuator incrementally rotates the wheel, based on the type of coupling between the MAD and the wheel actuator, further comprises:
rotating the first clutch equivalent to an amount of rotation by the MAD, wherein the rotation of the first clutch is not transmitted to the fourth clutch, due to disengagement between the first clutch and the fourth clutch.

3. The scalable fluid delivery apparatus as claimed in claim 1, wherein, in the first kinematic pathway corresponding to the high linear resolution mode of the PDM, upon rotation of the second clutch and the third clutch, the third gear and the fourth clutch subsequently rotates via the second gear, wherein the rotation of the fourth clutch is not transmitted to the first clutch, due to disengagement between the fourth clutch and the first clutch.

4. The scalable fluid delivery apparatus as claimed in claim 1, wherein, in the second kinematic pathway corresponding to the low linear resolution mode of the PDM, the wheel actuator incrementally rotates the wheel, based on the type of coupling between the MAD and the wheel actuator further comprises:
rotating the second clutch equivalent to an amount of rotation by the wheel, wherein the rotation of the second clutch is not transmitted to the third clutch, due to disengagement between the second clutch and the third clutch.

5. The scalable fluid delivery apparatus as claimed in claim 1, wherein, in the second kinematic pathway corresponding to the low linear resolution mode of the PDM, upon rotation of the third gear and the fourth clutch, the third clutch, the first gear and the threaded rotatable shaft subsequently rotates via the second gear, wherein the rotation of the third clutch is not transmitted to the second clutch, due to disengagement between the third clutch and the second clutch.

6. The scalable fluid delivery apparatus as claimed in claim 1, wherein, the PDM is constrained by a sliding joint with a non-circular cross-section, wherein the non-circular cross-section of the sliding joint does not allow the sliding joint to rotate upon rotation of the threaded rotatable shaft or the third clutch, and only moves linearly.

7. The scalable fluid delivery apparatus as claimed in claim 1, wherein the third clutch is coupled to an encoder via a shaft independent of the secondary shaft, wherein the encoder enables confirmation or control of the amount of rotation of the third clutch or the threaded rotatable shaft.

8. The scalable fluid delivery apparatus as claimed in claim 1, wherein the MAD rotates to provide intermittent motion or continuous motion using at least one of the first kinematic pathway and the second kinematic pathway.

9. The scalable fluid delivery apparatus as claimed in claim 1, wherein the MAD and switching mechanism comprises at least one of a motor, a linear motor, a liner actuator, a Shape Memory Alloy (SMA) actuator, and a Solenoid.

10. The scalable fluid delivery apparatus as claimed in claim 1, wherein the wheel actuator is actuated using at least one of an Eccentric Cam (EC) coupled to a motor, a linear motor, a linear actuator, a Shape Memory Alloy (SMA) actuator, and a Solenoid.

11. The scalable fluid delivery apparatus as claimed in claim 1, wherein the type of coupling between the MAD and the wheel actuator comprises an Eccentric Cam (EC).

12. A method for scalable fluid delivery comprising:
a first kinematic pathway step corresponding to a high linear resolution mode of a Piston Drive Member (PDM) comprising steps of:
receiving, by a scalable fluid delivery apparatus, an actuation from a switching mechanism to engage a second clutch and a third clutch, and to disengage a fourth clutch and a first clutch;
performing, by the scalable fluid delivery apparatus, an actuation by a Motion Actuating Device (MAD) to transmit motion to a wheel actuator, wherein the wheel actuator incrementally rotates a wheel, based on a type of coupling between the MAD and the wheel actuator;
rotating, by the scalable fluid delivery apparatus, the engaged second clutch and the third clutch equivalent to an amount of rotation of the wheel, wherein the third clutch and a threaded rotatable shaft rotate equivalent to an amount of rotation of the second clutch;
pushing, by the scalable fluid delivery apparatus, a plunger of a syringe by a distance corresponding to a lead of the threaded rotatable shaft and an angle of rotation of the third clutch, to create a linear motion at the Piston Drive Member (PDM); and
delivering, by the scalable fluid delivery apparatus, a fluid comprised in the syringe, upon pushing the plunger; and
the second kinematic pathway step corresponding to the low linear resolution mode of the PDM comprising steps of:
receiving, by the scalable fluid delivery apparatus, an actuation from the switching mechanism to disengage the second clutch and the third clutch, and to engage the first clutch and the fourth clutch;
performing, by the scalable fluid delivery apparatus, an actuation by the MAD to transmit a rotational motion to the wheel actuator, wherein the wheel actuator incrementally rotates the wheel, based on a type of coupling between the MAD and the wheel actuator;
rotating, by the scalable fluid delivery apparatus, the engaged fourth clutch and the first clutch equivalent to an amount of motion of the MAD;
transmitting, by the scalable fluid delivery apparatus, rotation of the fourth clutch to the third clutch, wherein the first gear, the third clutch and the threaded rotatable shaft rotates based on rotation transmitted from the second gear and the third gear, wherein the third clutch and the threaded rotatable shaft rotates equivalent to an amount of a gear ratio of the third gear and the first gear or to an amount of an arbitrary ratio based on a gear train configuration; and
performing, by the scalable fluid delivery apparatus, forward motion, or reverse motion of the PDM, corresponding to a lead and an angle of rotation of the third clutch and the threaded rotatable shaft, wherein the forward motion, or the reverse motion of the PDM is for priming the barrel and a removable fluid delivery tube or to revert the PDM to an initial position, respectively, or to deliver the fluid.

13. The method as claimed in claim 12, wherein, in the first kinematic pathway corresponding to the high linear resolution mode of the PDM, the wheel actuator incrementally rotates the wheel, based on the type of coupling between MAD and the wheel actuator, further comprises:
rotating, by the scalable fluid delivery apparatus, the first clutch equivalent to an amount of rotation by the MAD, wherein the rotation of the first clutch is not transmitted to the fourth clutch, due to disengagement between the first clutch and the fourth clutch.

14. The method as claimed in claim 12, wherein, in the first kinematic pathway corresponding to the high linear resolution mode of the PDM, upon rotation of the second clutch and the third clutch, the third gear and the fourth clutch subsequently rotates via the second gear, wherein the rotation of the fourth clutch is not transmitted to the first clutch, due to disengagement between the fourth clutch and the first clutch.

15. The method as claimed in claim 12, wherein, in the second kinematic pathway corresponding to the low linear resolution mode of the PDM, the wheel actuator incrementally rotates the wheel, based on the type of coupling between the MAD and the wheel actuator, further comprises:
rotating, by the scalable fluid delivery apparatus, the second clutch equivalent to an amount of rotation by the wheel, wherein the rotation of the second clutch is not transmitted to the third clutch, due to disengagement between the second clutch and the third clutch.

16. The method as claimed in claim 12, wherein, in the second kinematic pathway corresponding to the low linear resolution mode of the PDM, upon rotation of the third gear and the fourth clutch, the third clutch, the first gear and the threaded rotatable shaft subsequently rotates via the second gear, wherein the rotation of the third clutch is not transmitted to the second clutch, due to disengagement between the third clutch and the second clutch.

17. The method as claimed in claim 12, wherein, the PDM is constrained by a sliding joint with a non-circular cross-section, wherein the non-circular cross-section of the sliding joint does not allow the sliding joint to rotate upon rotation of the threaded rotatable shaft or the third clutch and only moves linearly.

18. The method as claimed in claim 12, wherein the third clutch is coupled to an encoder via a shaft independent of the secondary shaft, wherein the encoder enables confirmation or control of the amount of rotation of the third clutch or the threaded rotatable shaft.

19. The method as claimed in claim 12, wherein the MAD rotates to provide intermittent motion, or continuous motion using at least one of the first kinematic pathway and the second kinematic pathway.

20. The method as claimed in claim 12, wherein the MAD and the switching mechanism comprises at least one of a motor, a linear motor, a liner actuator, a Shape Memory Alloy (SMA) actuator, and a Solenoid.

21. The method as claimed in claim 12, wherein the wheel actuator is actuated using at least one of an Eccentric Cam (EC) coupled to a motor, a linear motor, a linear actuator, a Shape Memory Alloy (SMA) actuator, and a Solenoid.

22. The method as claimed in claim 12, wherein the type of coupling between the MAD and the wheel actuator comprises an Eccentric Cam (EC).

23. A scalable fluid delivery apparatus comprising a housing to accommodate a syringe containing/to be filled with a fluid, wherein the syringe comprises a barrel, a plunger within the barrel and a removable fluid delivery tube, the scalable fluid delivery apparatus comprising:
a driving unit removably coupled to the syringe, and operatively connected to a motor to advance the plunger of the syringe for expelling fluid from the barrel of the syringe, wherein the driving unit comprising:
a primary shaft coupled to the motor;
a secondary shaft;
a Compliant Ratchet Actuator (CRA) comprising a ratchet wheel mounted on the secondary shaft;
a plurality of clutches comprising a first clutch mounted on the primary shaft, a second clutch mounted on the secondary shaft, a third clutch mounted adjacent to the second clutch, and a fourth clutch mounted adjacent to the first clutch;
a threaded rotatable shaft rigidly coupled via a first end to the third clutch, and removably coupled via a second end to a Piston Drive Member (PDM), wherein the PDM linearly advances the plunger of the syringe when in contact, and during a linear motion of the PDM based on a lead of the threaded rotatable shaft and rotation of the third clutch;
a plurality of gears comprising a first gear rigidly coupled to the third clutch, a third gear coupled to the fourth clutch, a second gear coupled to the first gear and the third gear;
a switching mechanism coupled to the first clutch and the second clutch;
wherein the motor is configured to transmit motion in a plurality of pathways corresponding to a plurality of resolutions, wherein the plurality of pathways comprises at least one of a first kinematic pathway corresponding to a high linear resolution mode of the PDM and a second kinematic pathway corresponding to a low linear resolution mode of the PDM,
a first kinematic pathway corresponding to a high linear resolution mode of a Piston Drive Member (PDM) comprising steps of:
receiving an actuation from a switching mechanism to engage a second clutch and a third clutch, and to disengage a fourth clutch and a first clutch;
performing an actuation by a motor to transmit a rotational motion to the CRA via an Eccentric Cam (EC) attached to a primary shaft, wherein the CRA incrementally rotates the ratchet wheel, based on an eccentricity of the EC;
rotating the engaged second clutch and the third clutch, equivalent to an amount of rotation of the ratchet wheel, wherein the third clutch and a threaded rotatable shaft rotate equivalent to an amount of rotation of the second clutch;
pushing a plunger of a syringe by a distance corresponding to a lead of the threaded rotatable shaft and angle of rotation of the third clutch, to create a linear motion at the PDM; and
delivering a fluid comprised in the syringe, upon pushing the plunger; and
the second kinematic pathway corresponding to a low linear resolution mode of the PDM comprising steps of:
receiving an actuation from the switching mechanism to disengage the second clutch and the third clutch, and to engage the first clutch and the fourth clutch;
performing an actuation by the motor to transmit a rotational motion to a Compliant Ratchet Actuator (CRA) via the EC attached to the primary shaft, wherein the CRA incrementally rotates the ratchet wheel, based on the eccentricity of the EC;
rotating the engaged fourth clutch and the first clutch equivalent to an amount of rotation of the motor;
transmitting rotation of the fourth clutch to the third clutch, wherein the first gear, the third clutch and the threaded rotatable shaft rotates based on rotation transmitted from the second gear and the third gear, wherein the third clutch and the threaded rotatable shaft rotates equivalent to an amount of a gear ratio of the third gear and the first gear or to an amount of an arbitrary ratio based on a gear train configuration; and
performing forward or reverse motion of the PDM, corresponding to a lead and an angle of rotation of the third clutch and the threaded rotatable shaft, wherein the forward motion, or the reverse motion of the PDM is for priming a barrel of a syringe and a removable fluid delivery tube or to revert the PDM to an initial position, respectively or to deliver the fluid.

24. A scalable fluid delivery apparatus as claimed in claim 1 further comprises at least one of a one-way bearing or a clutch bearing within the primary shaft to restrict the motion transmitted to the wheel actuator.
